(19) United States
(12) Patent Application Publication
(10) Pub. No.: US 2024/0127050 A1
(43) Pub. Date: Apr. 18, 2024

(54) HIGH DIMENSIONAL AND ULTRAHIGH DIMENSIONAL DATA ANALYSIS WITH KERNEL NEURAL NETWORKS

(71) Applicant: University of Florida Research Foundation, Inc., Gainesville, FL (US)

(72) Inventors: Qing LU, Gainesville, FL (US); Xiaoxi SHEN, Gainesville, FL (US); Tingting HOU, Gainesville, FL (US)

(21) Appl. No.: 18/267,184

(22) PCT Filed: Dec. 8, 2021
(86) PCT No.: PCT/US2021/072811

§ 371 Date: Jun. 14, 2023

Related U.S. Application Data
(60) Provisional application No. 63/124,981, filed on Dec. 14, 2020.

(51) Int. Cl. G06N 3/08; G06N 5/022

(52) U.S. Cl. CPC G06N 3/08; G06N 5/022

(57) ABSTRACT

Various examples are provided related to the application of a kernel neural network (KNN) to the analysis of high dimensional and ultrahigh dimensional data for, e.g., risk prediction. In one embodiment, a method includes training a KNN with a training set to produce a trained KNN model, determining a likelihood of a condition based at least in part upon an output indication of the trained KNN corresponding to one or more phenotypes, identifying treatment or prevention strategy for an individual based at least in part upon the likelihood of the condition. The KNN model includes a plurality of kernels as a plurality of layers to capture complexity between the data with disease phenotypes. The training set of data includes genetic information applied as inputs to the KNN and the phenotype(s), and the output indication is based upon analysis of data comprising genetic information from the individual by the trained KNN.

### CROSS REFERENCE TO RELATED APPLICATIONS

This application claims priority to, and the benefit of, co-pending U.S. provisional application entitled “High-Dimensional and Ultrahigh Dimensional Data Analysis with Kernel Neural Networks” having Ser. No. 63/124,981, filed Dec. 14, 2020, which is hereby incorporated by reference in its entirety.

### STATEMENT REGARDING FEDERALLY SPONSORED RESEARCH OR DEVELOPMENT

This invention was made with government support under Grant Nos. R01 LM012848 and R01 DA043501, awarded by the National Institutes of Health. The government has certain rights in this invention.

### BACKGROUND

Early disease detection, prevention, and intervention are keys to public health. The concept of treating diseases with precise interventions, designed rationally from a detailed understanding of the disease etiology and individual differences, has been widely accepted as the goal of precision medicine. Including the human genome in disease prediction is an important step towards precision medicine. Genetic risk prediction is the cornerstone for genomic medicine, which holds great promise for early disease detection. While the large amounts of genetic variants generated from high-throughput technologies offer a unique opportunity to study a deep catalog of genetic variants for genetic risk prediction, the high-dimensionality of genetic data and complex relationships between genetic variants and disease outcomes bring tremendous challenges to risk prediction analysis. Moreover, the advance of genotyping technology and reduced cost have enabled the creation of bio-banks with hundreds of thousands of samples. These cohorts offered the material bases for exploring epistasis with sophisticated models and better prediction; on the other hand, such models are computationally difficult to train because of the large sample size. Advanced tools are in great need to address the analytical and computational challenges from ongoing risk prediction analysis.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic diagram illustrating an example of the hierarchical structure of a kernel-based neural network (KNN) model, in accordance with various embodiments of the present disclosure.

FIG. 2 Illustrates an example of the KNN path and linear mixed model (LMM) path, in accordance with various embodiments of the present disclosure.

FIGS. 3-5 illustrates examples of prediction errors for LMM and KNN, in accordance with various embodiments of the present disclosure.

FIGS. 6A and 6B illustrate examples of 2-layered NNT and KNN, in accordance with various embodiments of the present disclosure.

FIG. 7 illustrates examples of nonlinear functions, in accordance with various embodiments of the present disclosure.

FIGS. 8 and 9 illustrate comparisons of KNN and LMM simulation results, in accordance with various embodiments of the present disclosure.

FIG. 10 illustrates examples of batched training and leave one batch out (LOBO) testing, in accordance with various embodiments of the present disclosure.

FIGS. 11 and 12 illustrate examples of predictions of skin cancer and systolic blood pressure, in accordance with various embodiments of the present disclosure.

FIG. 13 is a schematic block diagram of an example of a computing device, in accordance with various embodiments of the present disclosure.

### SUMMARY

Aspects of the present disclosure are related to the application of kernel neural networks to analysis of high dimensional and ultrahigh dimensional data for, e.g., risk prediction, identification of treatment or prevention strategy, etc. In one aspect, among others, a method for risk prediction using high-dimensional and ultrahigh-dimensional data, comprising: training a kernel-based neural network (KNN) with a training set of data to produce a trained KNN model, the KNN model comprising a plurality of kernels as a plurality of layers to capture complexity between the data with disease phenotypes, the training set of data comprising genetic information applied as inputs to the KNN and one or more phenotypes; determining a likelihood of a condition based at least in part upon an output indication of the trained KNN corresponding to the one or more phenotypes, the output indication based upon analysis of data comprising genetic information from an individual by the trained KNN; and identifying a treatment or prevention strategy for the individual based at least in part upon the likelihood of the condition.

In one or more aspects, a first layer of the plurality of layers can comprise a plurality of kernels and a last layer of the plurality of layers can comprise a single kernel or a plurality of kernels. The plurality of kernels in the first layer can convert a plurality of data inputs into a plurality of latent variants. The plurality of data inputs can comprise single-nucleotide polymorphisms (SNPs) or biomarkers. Individual latent variables of the plurality of kernels can be generated by random sampling of outputs of the plurality of kernels. The single kernel or plurality of kernels of the last layer can determine the output indication based upon a plurality of latent variable produced by a preceding layer of the plurality of layers. The preceding layer can be the first layer. In various aspects, the KNN can be trained using minimum norm quadratic estimation. Training of the KNN can be accelerated using batch training.

In another aspect, a system for risk prediction comprises at least one computing device comprising processing circuitry including a processor and memory. The at least one computing device can be configured to at least: train a kernel-based neural network (KNN) with a training set of data to produce a trained KNN model, the KNN model comprising a plurality of kernels as a plurality of layers to capture complexity between the data with disease phenotypes, the training set of data comprising genetic information applied as inputs to the KNN and one or more phenotypes; determine a likelihood of a condition based at least in part upon an output indication of the trained KNN corresponding to the one or more phenotypes, the output indication based upon analysis of data comprising genetic information from an individual by the trained KNN; and identify a treatment or prevention strategy for the individual based at least in part upon the likelihood of the condition. In one or more aspects, the KNN can be trained using minimum norm quadratic estimation. Training of the KNN can be accelerated using batch training.

In various aspects, a first layer of the plurality of layers can comprise a plurality of kernels and a last layer of the plurality of layers can comprise a single kernel or a plurality of kernels. The plurality of kernels in the first layer can convert a plurality of data inputs into a plurality of latent variants. The plurality of data inputs can comprise single-nucleotide polymorphisms (SNPs) or biomarkers. Individual latent variables of the plurality of kernels can be generated by random sampling of outputs of the plurality of kernels. The single kernel or plurality of kernels of the last layer can determine the output indication based upon a plurality of latent variable produced by a preceding layer of the plurality of layers. The preceding layer can be the first layer.

In another aspect, a non-transitory computer-readable medium embodies a program executable in at least one computing device. When executed the program can cause the at least computing device to at least: train a kernel-based neural network (KNN) with a training set of data to produce a trained KNN model, the KNN model comprising a plurality of kernels as a plurality of layers to capture complexity between the data with disease phenotypes, the training set of data comprising genetic information applied as inputs to the KNN and one or more phenotypes; determine a likelihood of a condition based at least in part upon an output indication of the trained KNN corresponding to the one or more phenotypes, the output indication based upon analysis of data comprising genetic information from an individual by the trained KNN; and identify a treatment or prevention strategy for the individual based at least in part upon the likelihood of the condition.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

Disclosed herein are various examples related to the application of kernel neural networks and their application to the analysis of high dimensional and ultrahigh dimensional data. To address these rising challenges, a kernel-based neural network (KNN) method can be used. The KNN includes features from both linear mixed models (LMM) and classical neural networks and is designed for high-dimensional and ultrahigh dimensional risk prediction analysis. To deal with datasets with millions of variants, KNN summarizes genetic data into kernel matrices and uses the kernel matrices as inputs. Based on the kernel matrices, KNN can build a single-layer feedforward neural network, which makes it feasible to consider complex relationships between genetic variants and disease outcomes.

The parameter estimation in KNN can be based on minimum norm quadratic estimation (MINQUE) and, under certain conditions, the average prediction error of KNN can be smaller than that of LMM. For example, the computational problem can be solved with a close formed MINQUE coupled with a batched trained strategy. The advantages of KNN in prediction and the high speed of training by batched-MINQUE, via extensive simulation studies and by testing 43 phenotypes of nearly 400,000 samples from UK Biobank (UKB) can be demonstrated. Simulation studies also confirm the results. Reference will now be made in detail to the description of the embodiments as illustrated in the drawings, wherein like reference numbers indicate like parts throughout the several views.

Linear mixed effect models are powerful tools to model complex data structures. By adding random effects into the model, it becomes feasible to model correlated observations. Moreover, it is also possible to use linear mixed models to make the best predictions on the random effects. In genetic studies, more advantages of linear mixed models have been explored. For instance, in genome-wide association studies (GWAS), a simple linear regression can be conducted on each single-nucleotide polymorphism (SNP) so that there are a large number of hypotheses to be tested. The multiple test correction issue will also need to be dealt with. On the other hand, if the genetic effect is considered as a random effect, the null hypothesis may be reduced to testing whether the variance component of the random effect is zero or not. Applications can include: in sequence kernel association test (SKAT), a score type test based on a mixed effect model can be used to test the overall genetic effect; and genome-wide complex trait analysis (GCTA), which is also based on the linear mixed model, to address the “missing heretability” problem.

A kernel neural network (KNN) can be used for high-dimensional risk prediction analysis. Under certain scenarios, the model can be reduced to a linear mixed model. The KNN inherits an important property (i.e., considering nonlinear effects) from the neural network. Due to the complex structure of such method, it is difficult to obtain estimators for the parameters in the model. Moreover, it is difficult to obtain the marginal distribution of the response. To address these issues, instead of using the popular likelihood type inference using the restricted maximum likelihood estimator (REML), the minimum quadratic unbiased estimator (MINQUE) can be used to estimate the “variance components.” A basic description of the KNN and the estimation procedure for the parameters will be presented, including how to make predictions using KNN, followed by simulation results.

Kernel methods can be used in machine learning due to their capability of capturing nonlinear features from the data so that the prediction error can be reduced. Given a kernel and a training set, the kernel matrix can act as an information bottleneck, as all the information available to a kernel algorithm is extracted from this matrix. Therefore, neural networks can be integrated into the linear mixed model for genetic risk prediction. The covariance matrix of the random effect in the linear mixed model is a kernel matrix. For instance, consider the following linear mixed model:

y=Xβ+g+ϵ,

where y∈ [symbol] ⁿ is a vector of phenotypes; X is the design matrix for fixed effects β.

Start by creating latent features from the kernel matrix. Based on these latent features, higher order kernel matrices can be constructed. Consider that the phenotype y is modeled as a random effect model: given u₁, . . . , uₘ,

$$y \vert a , {u}_{1} , … , {u}_{m} ∼ {𝒩}_{n} ( a , ϕ {I}_{n} )$$

$$a \vert {u}_{1} , … , {u}_{m} ∼ {𝒩}_{n} ( 0 , {\sum }_{j = 1}^{J} {τ}_{j} {K}_{j} ( U ) ) ,$$

that is the covariance matrix of the random effect a depends on latent variables u₁, . . . , uₘ, Moreover, the latent variable uᵢ is modeled as follow:

$${u}_{1} , … , {u}_{m} ∼ i . i . d . {𝒩}_{n} ( 0 , {\sum }_{l = 1}^{L} {ξ}_{l} {K}_{l} ( X ) ) ,$$

where in the model, n is the sample size; m is the number of hidden units in the network; Kⱼ(U), j=1, . . . ,J are n×n are kernel matrices constructed based on the hidden nodes and Kₗ(U), l=1, . . . ,l are kernel matrices constructed based on the genotype matrices. Also define U=[u₁ . . . uₘ]∈ [symbol] ^{n×m}.

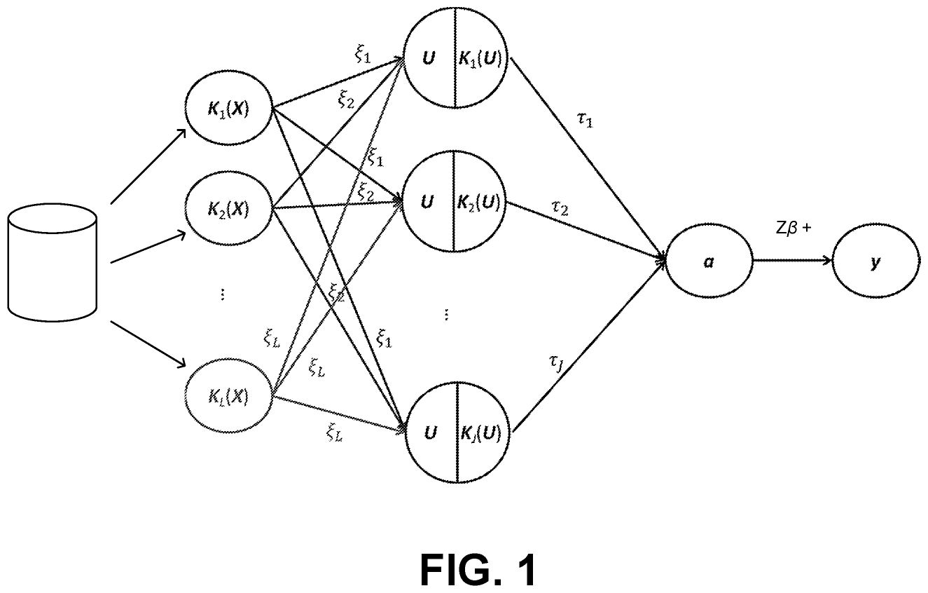

The basic hierarchical structure of the model is illustrated in FIG. 1. Due to the similarity in the network structures of KNN and neural networks, the model is called kernel neural network (KNN). Nonetheless, KNN uses kernel matrices as inputs, while the classic neural network uses the original data X as inputs.

Quadratic Estimators for Variance Components. Popular estimation strategies for variance components in linear models are the maximum likelihood estimator (MLE) and the restricted maximum likelihood estimator (REML). However, both methods depend on the marginal distribution of y. In the kernel neural network (KNN) model, it is generally difficult to obtain the marginal distribution of y, which involves high dimensional integration with respect to u₁, . . . , uₘ. Moreover, the uᵢ's are embedded in the kernel matrix K(U), which makes the integration even more complicated.

On the other hand, given the model described in the previous paragraph, it can be known that:

$$y \vert {u}_{1} , … , {u}_{m} ∼ {𝒩}_{n} ( 0 , {\sum }_{j = 1}^{J} {τ}_{j} {K}_{j} ( U ) + ϕ {I}_{n} ) .$$

Then the marginal mean and variance of y can be obtained via conditioning arguments:

$$\begin{matrix}𝔼 [ y ] = 𝔼 ( 𝔼 [ y \vert {u}_{1} , … , {u}_{m} ] ) = 0. \\ Var [ y ] = 𝔼 [ Var ( y \vert {u}_{1} , … , {u}_{m} ) ] + Var [ 𝔼 ( y \vert {u}_{1} , … , {u}_{m} ) ] \\ = 𝔼 [ {\sum }_{j = 1}^{J} {τ}_{j} {K}_{j} ( U ) + ϕ {I}_{n} ] \\ = {\sum }_{j = 1}^{J} {τ}_{j} 𝔼 [ {K}_{j} ( U ) ] + ϕ {I}_{n} . \\ := {\sum }_{j = 1}^{J} {τ}_{j} 𝔼 [ {K}_{j} ( U ) ] ,\end{matrix}$$

where τ₀=ϕ and K₀(U)=Iₙ. Given the marginal mean and variance of y, the minimum quadratic unbiased estimator (MINQUE) can be used. The basic idea of MINQUE is to use a quadratic form y^{T}Θy to estimate a linear combination of variance components. The MINQUE matrix Θ is obtained by minimizing a suitable matrix norm, which is typically chosen to be the Frobenius norm, of the difference between Θ and the matrix in the quadratic estimator by assuming that the random components in the linear models are known. The constraint in the optimization problem is the unbiasedness condition. One advantage of MINQUE is that it has a closed form solution provided by Lemma 3.4 in “Estimation of variance and covariance componentsminque theory” by C. R. Rao (Journal of multivariate analysis 1(3), pp. 257-275, 1971) so that it can be computed efficiently. However, MINQUE can also provide a negative estimate for a single variance component. To address this issue, consider a modified MINQUE (MINQUE). The idea is that we first obtain the original MINQUE and then we project the MINQUE matrix Θ onto the positive semi-definite cone S₊ⁿ. This can be accomplished by first doing a spectral decomposition of Θ, and then replace the negative eigenvalues of Θ by 0, that is the MINQUE matrix \hat{Θ} is obtained as follows:

$$\hat{Θ} = Q [ \begin{matrix}max \{ {λ}_{1} , 0 \} & & \\ & ⋱ & \\ & & max \{ {λ}_{n} , 0 \}\end{matrix} ] {Q}^{T} ,$$

where Q_{diag}{λ₁, . . . ,λₙ}Q^{T} is the spectral decomposition of Θ. The MINQUE for the linear combination of variance components is then obtained by y^{T}\hat{Θ}y.

MINQUE in KNN. For the ease of theoretical justifications, focus on the case where j=1 and the elements of the kernel matrix U are of the form

$${K}_{i j} ( U ) = f [ \frac{1}{m} {w}_{i}^{T} {w}_{j} ]$$

and w₁, . . . , wₙ are the n rows of the matrix U or some function f.

Start by considering the simplest case f(x)=x, in which case, the kernel matrix K(U) in KNN becomes

$$K ( U ) = \frac{1}{m} U {U}^{T} .$$

In this case, an explicit form of the marginal variance of y can be obtained. Since UU^{T}˜ [symbol] ₙ(m, Σₗ₌₁^{L}ξₗKₗ(X)), then:

$$V := Var [ y ] = τ𝔼 [ K ( U ) ] + ϕ {I}_{n} = {\sum }_{l = 1}^{L} {τξ}_{l} {K}_{l} ( X ) + ϕ {I}_{n} . \qquad ( 1 )$$

From equation (1), there is an identifiability issue when performing estimation procedure directly on τ and ξₗ. To resolve this issue, we can reparameterize the equation by letting θₗ=τξₗ, Θ₀=ϕ, K₀(X) =Iₙ and rewrite Var[y] as:

$$V = {\sum }_{l = 1}^{L} {θ}_{l} {K}_{l} ( X ) = {\sum }_{l = 1}^{L} {θ}_{l} {S}_{l} ( X ) {S}_{l}^{T} ( X ) , \qquad ( 2 )$$

where S₀(X), . . . ,S_{L}(X) are the Cholesky lower triangles for the kernel matrices K₀(X), . . . ,K_{L}(X), respectively. Then the parameters θ₀, θ₁, . . . , θ_{L} can be estimated via MINQUE.

For general kernel matrix of the form

$$K ( U ) = f [ \frac{1}{m} {UU}^{T} ] ,$$

where f[B] means applying the map f: [symbol] → [symbol] element-wisely to the matrix B and f is a function on [symbol] satisfying the following property, called the Generalized Linear Separable Condition:

$$f ( {\sum }_{α = 1}^{k} {c}_{α} {x}_{α} ) = {\sum }_{α = 1}^{{k}^{′}} {g}_{o} ( {c}_{1} , … , {c}_{k} ) {h}_{α} ( {x}_{1} , … , {x}_{k} ) , \qquad ( 3 )$$

where c₁, . . . , cₖ∈ [symbol] are coefficients and g₁, . . . , gₖ. . . , h₁, . . . , hₖare some functions. Common examples of such type of kernel functions are polynomial kernels. It is known that:

$${K}_{st} ( U ) = f ( \frac{{w}_{s}^{T} {w}_{t}}{m} ) , s , t = 1 , … , n and \qquad ( 4 )$$

$$[ \begin{matrix}{w}_{i 1} \\ {w}_{j 1}\end{matrix} ] , … , [ \begin{matrix}{w}_{i m} \\ {w}_{j m}\end{matrix} ] ∼ i . i . d . {𝒩}_{2} ( [ \begin{matrix}0 \\ 0\end{matrix} ] , [ \begin{matrix}{σ}_{ii} & {σ}_{ij} \\ {σ}_{ij} & {σ}_{jj}\end{matrix} ] ) , \qquad ( 5 )$$

where σᵢᵢ, σⱼⱼ, σᵢⱼ are corresponding elements Σ=Σₗ₌₁^{L}ξₗKₗ(X). The idea now is to use Taylor expansion to obtain an approximation of [symbol] [Kᵢⱼ(U)], which is show in detail in Lemma 1.

Lemma 1. Let the random vectors wᵢ, wⱼ∈ [symbol] ^{m} be as in equation (5) and the Kᵢⱼ(U) as defined in equation (4). Then if:

$$\vert {f}^{H} ( {λσ}_{ij} + ( 1 - λ ) \frac{{w}_{i}^{T} {w}_{j}}{m} ) \vert \leq M , a . s . , \qquad ( 6 )$$

for some M>0 and all λ∈[0,1], we have:

$$ℙ ( \vert {K}_{ij} ( U ) - {\hat{K}}_{ij} ( U ) \vert > δ ) \leq 4 exp \{ - m ( 1 ∧ \frac{δ}{20 {Ms}_{ij}} ∧ \frac{δ}{M {σ}_{ij}^{2}} ∧ \frac{1}{4 \vert {σ}_{ij} \vert} \sqrt{\frac{2 δ}{M}} ) \} ,$$

$$where {\hat{K}}_{ij} ( U ) = f ( {σ}_{ij} ) + {f}^{′} ( {σ}_{ij} ) ( \frac{{w}_{i}^{T} {w}_{j}}{m} - {σ}_{ij} ) .$$

Proof. Note that:

$$𝔼 [ \frac{{w}_{i}^{T} {w}_{j}}{m} ] = \frac{1}{m} {\sum }_{k = 1}^{m} 𝔼 [ {w}_{ik} {w}_{jk} ] = {σ}_{ij} .$$

Now consider the Taylor expansion of Kᵢⱼ(U) around σᵢⱼ:

$${K}_{ij} ( U ) = f ( {σ}_{ij} ) + {f}^{′} ( {σ}_{ij} ) ( \frac{{w}_{i}^{T} {w}_{j}}{m} - {σ}_{ij} ) + \frac{1}{2} {f}^{″} ( {η}_{ij} ) {( \frac{{w}_{i}^{T} {w}_{j}}{m} - {σ}_{ij} )}^{2} ,$$

where ηᵢⱼ is between σᵢⱼ and

$$\frac{{w}_{i}^{T} {w}_{j}}{m} .$$

Then the truncation error can be evaluated as follows. For all δ>0.

$$\begin{matrix}ℙ ( \vert {K}_{i , j} ( U ) - {\hat{K}}_{ij} ( U ) \vert > δ ) = ℙ ( \frac{1}{2} \vert {f}^{″} ( {η}_{ij} ) \vert {( \frac{{w}_{i}^{T} {w}_{j}}{m} - {σ}_{ij} )}^{2} > δ ) \leq \\ ℙ ( \frac{1}{2} {M ( \frac{{w}_{i}^{T} {w}_{j}}{m} - {σ}_{ij} )}^{2} > δ ) \\ = ℙ ( \vert \frac{{w}_{i}^{T} {w}_{j}}{m} - {σ}_{ij} \vert > \sqrt{\frac{2 δ}{M}} ) .\end{matrix}$$

So it suffices to evaluate the tail probability

$$ℙ ( \vert \frac{{w}_{i}^{T} {w}_{j}}{m} - {σ}_{ij} \vert > \sqrt{\frac{2 δ}{M}} ) .$$

Note that:

$${w}_{j} \vert {w}_{i} ∼ {𝒩}_{m} ( \frac{{σ}_{ij}}{{σ}_{ii}} {w}_{i} , ( {σ}_{jj} - \frac{{σ}_{ij}^{2}}{{σ}_{ii}} ) {I}_{m} ) ,$$

$$then : {w}_{i}^{T} {w}_{j} \vert {w}_{i} ∼ 𝒩 ( \frac{{σ}_{ij}}{{σ}_{ii}} {w}_{i}^{T} {w}_{i} , ( {σ}_{jj} - \frac{{σ}_{ij}^{2}}{{σ}_{ii}} ) {w}_{i}^{T} {w}_{i} ) .$$

Therefore, given wᵢ, the random variable wᵢ^{T}wⱼ is sub-Gaussian with sub-Gaussian parameter σᵢᵢ⁻¹sᵢⱼwᵢ^{T}wⱼ, where sᵢⱼ=σᵢᵢσⱼⱼ−σᵢᵢ². Since:

$$ℙ ( \vert \frac{{w}_{i}^{T} {w}_{j}}{m} - {σ}_{ij} \vert > \sqrt{\frac{2 δ}{M}} ) \leq ℙ ( \vert \frac{{w}_{i}^{T} {w}_{j}}{m} - \frac{{σ}_{ij}}{{σ}_{ii}} \frac{{w}_{i}^{T} {w}_{j}}{m} \vert > \frac{1}{2} \sqrt{\frac{2 δ}{M}} ) + ℙ ( \vert \frac{{σ}_{ij}}{{σ}_{ii}} \frac{{w}_{i}^{T} {w}_{j}}{m} - {σ}_{ij} \vert > \frac{1}{2} \sqrt{\frac{2 δ}{M}} ) := ( I ) + ( II ) ,$$

It suffices to provide bounds for (I) and (II).
For (II), note that wᵢ˜ [symbol] ₘ(0,σᵢᵢIₘ), having

$$\frac{1}{{σ}_{i i}} {w}_{i}^{T} {w}_{i} ∼ {χ}_{m}^{2} ,$$

which implies that:

$$\begin{matrix}𝔼 [ {e}^{λ ( \frac{{v}_{i}^{T} {v}_{i}}{{e}_{ii}} - m )} ] = {e}^{- λ m} 𝔼 [ {e}^{λ \frac{{v}_{i}^{T} {v}_{i}}{{e}_{ii}}} ] & \\ = {{e}^{- λ m} ( 1 - 2 λ )}^{- \frac{m}{2}} , & for λ < \frac{1}{2} \\ = {( \frac{{e}^{- λ}}{\sqrt{1 - 2 λ}} )}^{m} \leq & \\ {e}^{2 m {λ}^{2}} , & for all \vert λ \vert < \frac{1}{4} \\ = {e}^{\frac{4 m {λ}^{2}}{2}} , & for all \vert λ \vert < \frac{1}{4} ,\end{matrix}$$

i.e.,

$$\frac{1}{{σ}_{i i}}$$

wᵢ^{T}wᵢ is sub-exponential with parameters (2\sqrt{m}, 4) and hence for σᵢⱼ≠0, by Theorem 5, then:

$$\begin{matrix}( II ) = ℙ ( \vert \frac{1}{{σ}_{ii}} {w}_{i}^{T} {w}_{i} - m \vert > \frac{2 m}{\vert {σ}_{ij} \vert} \sqrt{\frac{2 δ}{M}} ) \\ \leq 2 exp \{ - ( \frac{δ m}{{σ}_{ij}^{2} M} ∧ \frac{m}{4 \vert {σ}_{ij} \vert} \sqrt{\frac{2 δ}{M}} ) \}\end{matrix} . \qquad ( 7 )$$

If σᵢⱼ=0, then clearly (II)=0.

For (I), by Hoeffding inequality, then:

$$\begin{matrix}( I ) = {𝔼}_{{w}_{i}} [ ℙ ( \vert \frac{{w}_{i}^{T} {w}_{j}}{m} - \frac{{σ}_{ij}}{{σ}_{ii}} \frac{{w}_{i}^{T} {w}_{i}}{m} \vert > \frac{1}{2} \sqrt{\frac{2 δ}{M}} \vert {w}_{i} ) ] \\ = {𝔼}_{{w}_{i}} [ ℙ ( \vert \frac{{w}_{i}^{T} {w}_{j}}{m} - \frac{{σ}_{ij}}{{σ}_{ii}} {w}_{i}^{T} {w}_{i} \text{\textbar >} \frac{m}{2} \sqrt{\frac{2 δ}{M}} \vert {w}_{i} ) ] \\ \leq {𝔼}_{{w}_{i}} [ 2 exp \{ - \frac{{σ}_{ii} {m}^{2} δ}{4 {Ms}_{ij} {w}_{i}^{T} {w}_{i}} \} ]\end{matrix} \qquad ( 8 )$$

Following from Lemma 1 in “Adaptive estimation of a quadratic functional by model selection” by B. Laurent and P. Massart (Annals of Statistics, pp. 1302-1338, 2000), Theorem A in “Inequalities for quantiles of the chi-square distribution” by T. Inglot (Probability and Mathematical Statistics, 30(2), pp. 339-351, 2010) stated that for a random variable χ˜χₘ², the 100(1−α)th percentile is upper bounded by m+log(1/α)+2\sqrt{m log(1/α)}, which is of the order [symbol] (m) as m→∞. Now, since σᵢᵢ⁻¹wᵢ^{T}wᵢ˜χ²ₘ, then for any α∈(0,1):

$$ℙ ( {σ}_{ii}^{- 1} {w}_{i}^{T} {w}_{i} \geq m + 2 log \frac{1}{α} + 2 \sqrt{2 m log \frac{1}{α}} ) = α .$$

Let q(α, m)=m+2 log(1/α)+2\sqrt{2m log(1/α)}. Since the function exp{−a/x} is increasing in x for a>0, equation (8) can be further bound as follows:

$$\begin{matrix}( I ) \leq {𝔼}_{{w}_{i}} [ 2 exp \{ - \frac{{m}^{2} δ}{4 {Ms}_{ij} {σ}_{ii}^{- 1} {w}_{i}^{T} {w}_{i}} \} {𝕀}_{\{ {σ}_{ii}^{- 1} {w}_{i}^{T} {w}_{i} \leq q ( α , m ) \}} ] + \\ {𝔼}_{{w}_{i}} [ 2 exp \{ - \frac{{m}^{2} δ}{4 {Ms}_{ij} {σ}_{ii}^{- 1} {w}_{i}^{T} {w}_{i}} \} {𝕀}_{\{ {σ}_{ii}^{- 1} {w}_{i}^{T} {w}_{i} \geq q ( α , m ) \}} ] \\ \leq 2 exp \{ - \frac{{m}^{2} δ}{4 {Ms}_{ij} q ( α , m )} \} + 2 ℙ ( {σ}_{ii}^{- 1} {w}_{i}^{T} {w}_{i} \geq q ( α , m ) ) \\ \leq 2 exp \{ - \frac{{m}^{2} δ}{4 {Ms}_{ij} q ( α , m )} \} + 2 α\end{matrix}$$

By choosing m=exp{−m}, then one gets q(α,m)=m+2m+2m+2\sqrt{2m²} so that:

$$\begin{matrix}( I ) \leq 2 exp \{ - \frac{{m}^{2} δ}{20 {Mms}_{ij}} \} + 2 exp \{ - m \} \\ \leq 2 exp \{ - m ( 1 ∧ \frac{δ}{20 {Ms}_{ij}} ) \} .\end{matrix} \qquad ( 9 )$$

Combining equations (9) and (7), then for all δ>0:

$$\begin{matrix}ℙ ( \vert {K}_{ij} ( U ) - {\hat{K}}_{ij} ( U ) \vert > δ ) \leq ( I ) + ( II ) \\ \leq 4 exp \{ - m ( 1 ∧ \frac{δ}{20 {Ms}_{ij}} ∧ \frac{δ}{M {σ}_{ij}^{2}} ∧ \frac{1}{4 \vert {σ}_{ij} \vert} \sqrt{\frac{2 δ}{M}} ) \} .\end{matrix}$$

Remark 1. The condition of equation (6) can be weakened as follows:

$${f}^{″} ( {λσ}_{ij} + ( 1 - λ ) \frac{{w}_{i}^{T} {w}_{j}}{m} ) = {𝒪}_{p} ( 1 )$$

for all λ∈[0,1]. In such case, the evaluation of truncation error can be modified as follows. For all δ>0, there exists M_{δ}>0 such that:

$$ℙ ( \vert {f}^{″} ( {η}_{ij} ) \vert > {M}_{δ} ) < \frac{δ}{2} ,$$

where

$${η}_{ij} = λ {σ}_{ij} + ( 1 - λ ) \frac{{w}_{i}^{T} {w}_{j}}{m}$$

for some λ∈[0,1] and then:

$$\begin{matrix}ℙ ( \vert {K}_{ij} ( U ) - {\hat{K}}_{ij} ( U ) \vert > δ ) = ℙ ( \frac{1}{2} {f}^{″} ( {η}_{ij} ) {( \frac{{w}_{i}^{T} {w}_{j}}{m} - {σ}_{ij} )}^{2} > δ ) \\ \leq ℙ ( \{ \frac{1}{2} {f}^{″} ( {η}_{ij} ) {( \frac{{w}_{i}^{T} {w}_{j}}{m} - {σ}_{ij} )}^{2} > δ \} ⋂ \\ \{ \vert {f}^{″} ( {η}_{ij} ) \vert \leq {M}_{δ} \} ) + \\ ℙ ( \vert {f}^{″} ( {η}_{ij} ) \vert > {M}_{δ} ) \\ \leq ℙ ( \vert \frac{{w}_{i}^{T} {w}_{j}}{m} - {σ}_{ij} \vert > \sqrt{\frac{2 δ}{{M}_{δ}}} + \frac{δ}{2} \\ \leq 4 exp \{ - m ( 1 ∧ \frac{δ}{20 {M}_{δ} {s}_{ij}} ∧ \frac{δ}{4 \vert {σ}_{ij} \vert} \sqrt{\frac{2 δ}{{M}_{δ}}} ) \} + \frac{δ}{2}\end{matrix}$$

Now, choose

$$m > {( 1 ∧ \text{?} )}^{- 1} log \text{?}$$

$$\text{?} \text{indicates text missing or illegible when filed}$$

so that:

$$ℙ ( \vert {K}_{ij} ( U ) - {\hat{K}}_{ij} ( U ) \vert > δ ) < \frac{δ}{2} + \frac{δ}{2} = δ$$

And hence Kᵢⱼ(U)=\hat{K}ᵢⱼ(U)+oₚ(1).

Lemma 1 and Remark 1 show that when \hat{K}(U)=[\hat{K}ᵢⱼ(U)] is used to approximate K(U), the approximation will be sufficiently small when the number of hidden units is large enough. Hence, K(U) can be written as follows:

$$K ( U ) = \hat{K} ( U ) + {o}_{P} ( 1 ) = f [ \sum ] + {f}^{′} [ \sum ] ⊙ ( \frac{1}{m} {UU}^{T} - \sum ) + {o}_{P} ( 1 ) , \qquad ( 10 )$$

where ⊙ means the Hadamard product of two matrices. Moreover, the Strong Law of Large Numbers implies

$$\frac{1}{m} U {U}^{T} \to \sum a . s .$$

Hence, equation (10) can be further written as:

K(U)=f[Σ]+o_{P}(1),

i.e.,

$$K ( U ) {\to }^{P} f \vert \sum \vert$$

as m→∞ element-wisely.

Lemma 2. Under the assumptions of Lemma 1, if

$${f}^{n} ( {η}_{ij} ) {( \frac{{w}_{i}^{T} {w}_{j}}{m} - {σ}_{ij} )}^{2} ∈ {L}^{1} ( ℙ ) , then ;$$

$$𝔼 [ \frac{1}{2} {f}^{n} ( {η}_{ij} ) {( \frac{{w}_{i}^{T} {w}_{j}}{m} - {σ}_{ij} )}^{2} o ( 1 ) ,$$

where

$${η}_{ij} = {λσ}_{ij} + ( 1 - λ ) \frac{{w}_{i}^{T} {w}_{j}}{m}$$

for some λ∈[0,1].
Proof. This follows from the version of Dominated Convergence Theorem using convergence in probability.

Based on Lemma 2, the marginal variance-covariance matrix V can be written as:

$$\begin{matrix}V = τ𝔼 [ K ( U ) ] + ϕ {I}_{n} \\ ≃ τ𝔼 [ \hat{K} ( U ) ] + ϕ {I}_{n} \\ ≃ τ f [ \sum ] + ϕ {I}_{n} \\ = τ {\sum }_{l = 1}^{{L}^{′}} {g}_{l} ( {ξ}_{1} , … , {ξ}_{L} ) {h}_{l} [ {K}_{1} ( X ) , … , {K}_{L} ( X ) ] + ϕ {I}_{n} \\ = {\sum }_{l = 0}^{L} {θ}_{l} {S}_{l} ( X ) {S}_{l}^{T} ( X ) ,\end{matrix}$$

where θ₀=ϕ, θₗ=τgₗ(ξ₁, . . . , ξ_{L}), l=1, . . . , L′ and S₀(X)=Iₙ, Sₗ(X) is the Cholesky lower triangle for the matrix hₗ[K₁(X), . . . ,K_{L}(X)]l=1, . . . ,L. Then the parameters θ₀, . . . , θ_{L} can be estimated via MINQUE as well. Based on the above discussion, it can be seen that the estimation of the variance components in KNN through MINQUE is an approximation. In this way, a complex mixed model is used to approximate KNN.

Predictions. Next, a comparison of predicted performance between KNN and LMM is made. Based on the disclosed model, the best predictor for a can be given by:

$$\begin{matrix}\hat{y} = 𝔼 [ a \vert y ] = 𝔼 [ 𝔼 ( a \vert y , {u}_{1} , … , {u}_{m} ) ] \\ = 𝔼 [ ( {\sum }_{j = 1}^{J} {τ}_{j} {K}_{j} ( U ) ) {( {\sum }_{j = 1}^{J} {τ}_{j} {K}_{j} ( U ) + ϕ {I}_{n} )}^{- 1} ] y \\ = 𝔼 [ ( {\sum }_{j = 1}^{J} {ϕ}^{- 1} {τ}_{j} {K}_{j} ( U ) ) {( {\sum }_{j = 1}^{J} {ϕ}^{- 1} {τ}_{j} {K}_{j} ( U ) + {I}_{n} )}^{- 1} ] y \\ := 𝔼 [ ( {\sum }_{j = 1}^{J} {\tilde{τ}}_{j} {K}_{j} ( U ) ) {( {\sum }_{j = 1}^{J} {\tilde{τ}}_{j} {K}_{j} ( U ) + {I}_{n} )}^{- 1} ] y ,\end{matrix}$$

where \tilde{τ}ⱼ=τⱼϕ⁻¹, j=1, . . . ,m. The prediction error based on ŷ is given by:

$$\begin{matrix}R = {( y - \hat{y} )}^{T} ( y - \hat{y} ) \\ = {{y}^{T} ( {I}_{n} - 𝔼 [ ( {\sum }_{j = 1}^{J} {\tilde{τ}}_{j} {K}_{j} ( U ) ) {( {\sum }_{j = 1}^{J} {\tilde{τ}}_{j} {K}_{j} ( U ) + {I}_{n} )}^{- 1} ] )}^{T} \\ ( {I}_{n} - 𝔼 [ ( {\sum }_{j = 1}^{J} {\tilde{τ}}_{j} {K}_{j} ( U ) ) {( {\sum }_{j = 1}^{J} {\tilde{τ}}_{j} {K}_{j} ( U ) + {I}_{n} )}^{- 1} ] ) y .\end{matrix}$$

Note that:

$${I}_{n} - 𝔼 [ ( {\sum }_{j = 1}^{J} {\tilde{τ}}_{j} {K}_{j} ( U ) ) {( {\sum }_{j = 1}^{J} {\tilde{τ}}_{j} {K}_{j} ( U ) + {I}_{n} )}^{- 1} ] = 𝔼 [ ( {\sum }_{j = 1}^{J} {\tilde{τ}}_{j} {K}_{j} ( U ) + {I}_{n} - {\sum }_{j = 1}^{J} {\tilde{τ}}_{j} {K}_{j} ( U ) ) {( {\sum }_{j = 1}^{J} {\tilde{τ}}_{j} {K}_{j} ( U ) + {I}_{n} )}^{- 1} ] = 𝔼 {( {\sum }_{j = 1}^{J} {\tilde{τ}}_{j} {K}_{j} ( U ) + {I}_{n} )}^{- 1} ] ,$$

### Thus,

$$R = {{y}^{T} ( 𝔼 [ {( {\sum }_{j = 1}^{J} {\tilde{τ}}_{j} {K}_{j} ( U ) + {I}_{n} )}^{- 1} ] )}^{2} y .$$

Direct evaluation of the prediction error R is complicated. Instead, it can be approximated through some asymptotic results. Consider the case where J=1 and

$$K ( U ) = f [ \frac{1}{m} {UU}^{T} ] .$$

Lemma 3 (Approximation of Prediction Error). (i) When f(x)=x, then as m→∞,

$$R ≃ {{y}^{T} ( {\sum }_{l = 1}^{L} \tilde{τ} ξ {K}_{l} ( X ) + {I}_{n} )}^{- 2} y .$$

(ii) When f is continuous and f|Σ|∈ [symbol] ₊ⁿ, then as m→∞,

$$R ≃ {{y}^{T} ( \tilde{τ} f [ {\sum }_{l = 1}^{L} ξ {K}_{l} ( X ) ] + {I}_{n} )}^{- 2} y .$$

Proof. (i) when f(x)=x, then

$$K ( U ) = \frac{1}{m} {UU}^{T}$$

and due to the i.i.d. assumption on the hidden random vectors u₁, . . . , uₘ, it can be known that:

$${u}_{1} {u}_{1}^{T} , … , {u}_{m} {u}_{m}^{T} ∼ i . i . d . {𝒲}_{n} ( 1 , {\sum }_{l = 1}^{L} {ξ}_{l} {K}_{l} ( X ) ) ,$$

where [symbol] ₙ(1, Σₗ₌₁^{L}ξₗKₗ(X)) stand for a Wishart distribution with degrees of freedom 1 and covariance matrix Σₗ₌₁^{L}ξₗKₗ(X). Therefore, the Strong Law of Large Numbers implies that as m→∞.

$$K ( U ) = \frac{1}{m} {UU}^{T} = \frac{1}{m} {\sum }_{i = 1}^{m} {u}_{i} {u}_{i}^{T} \to 𝔼 [ {u}_{1} {u}_{1}^{T} ] = {\sum }_{l = 1}^{L} {ξ}_{l} {K}_{l} ( X ) , a . s ..$$

Since the matrix inverse map is continuous, then by the continuous mapping theorem, one can obtain:

$${( \tilde{τ} K ( U ) + {I}_{n} )}^{- 1} \to {( {\sum }_{l = 1}^{L} \tilde{τ} ξ {K}_{l} ( X ) + {I}_{n} )}^{- 1} , a . s . , as m \to ∞ .$$

Let Ä=(\ddot{τ}K(U)+Iₙ)⁻¹, then:

$$\begin{matrix}{max}_{1 \leq i , j \leq n} \vert {\tilde{A}}_{ij} \vert \leq {‖ \tilde{A} ‖}_{∞} ≾ {‖ \tilde{A} ‖}_{op} \\ = {λ}_{max} ( {( \tilde{τ} K ( U ) + {I}_{n} )}^{- 1} ) \\ = \frac{1}{\tilde{τ} {λ}_{min} ( K ( U ) ) + 1} \leq 1 < ∞ .\end{matrix}$$

Therefore, the bounded convergence theorem implies that:

$$A := 𝔼 [ {( \tilde{τ} K ( U ) + {I}_{n} )}^{- 1} ] \to {( {\sum }_{l = 1}^{L} \tilde{τ} ξ {K}_{l} ( X ) + {I}_{n} )}^{- 1} , a . s . , as m \to ∞$$

and asymptotically, then as m→∞

$$R ≃ {{y}^{T} ( {\sum }_{l = 1}^{L} \tilde{τ} ξ {K}_{l} ( X ) + {I}_{n} )}^{- 2} y .$$

(ii) Note that equation (10) can be further written as:

K(U)=f[Σ]+op(1)

or equivalently,

$$K ( U ) \to f \vert Σ \vert P$$

as m→∞ element-wisely. Similarly, under the assumption that ∥K(U)∥ₒ<∞ a.s., then:

[symbol]

[\ddot{τ}K(U)+Iₙ]→\ddot{τ}f[Σ]+Iₙ.

### Hence, by the Bounded Convergence Theorem and Continuous Mapping Theorem, one can get:

A= [symbol] [(\ddot{τ}K(U)+Iₙ)⁻¹]→(\ddot{τ}f[Σ]+Iₙ)⁻¹, as m→∞,

which shows that as m→∞,

$$R ≃ {{y}^{T} ( \tilde{τ} f [ {\sum }_{l = 1}^{L} ξ {K}_{l} ( X ) ] + {I}_{n} )}^{- 2} y .$$

Now, the average prediction error between kernel neural network (KNN) and linear mixed model (LMM) is compared. For an LMM, the prediction error using best predictor can be obtained as follows:

Proposition 1 (Prediction Error Using Linear Mixed Model). Consider the linear mixed effect model:

y=a+ϵ;

a˜ [symbol] (0, σ_{R}²Σ);

ϵ˜ [symbol] (0, ϕIₙ).

Then the prediction error under quadratic loss using the best predictor ŷ= [symbol] [a|y]=\tilde{σ}_{R}²Σ(\tilde{σ}_{R}²Σ+Iₙ)⁻¹y with \tilde{σ}_{R}²=\tilde{σ}_{R}²ϕ⁻¹ is given by:

$$APELMM = ϕ {\sum }_{i = 1}^{n} {( {\tilde{σ}}_{R}^{2} {λ}_{i} ( Σ ) + 1 )}^{- 1} ,$$

where APELMM stands for average prediction error for linear mixed model.
Proof. The desired result follows by noting that:

$$\begin{matrix}APELMM = 𝔼 [ {( y - 𝔼 [ a \vert y ] )}^{T} ( y - 𝔼 [ a \vert y ] ) ] \\ = 𝔼 [ {{y}^{T} ( {I}_{n} - {\tilde{σ}}_{R}^{2} {Σ ( {\tilde{σ}}_{R}^{2} Σ + {I}_{n} )}^{- 1} )}^{T} ( {I}_{n} - {\tilde{σ}}_{R}^{2} {Σ ( {\tilde{σ}}_{R}^{2} + {I}_{n} )}^{- 1} ) y ] \\ = 𝔼 [ {{y}^{T} ( {( {\tilde{σ}}_{R}^{2} Σ + {I}_{n} )}^{- 1} )}^{2} y ] \\ = tr [ {( {( {\tilde{σ}}_{R}^{2} Σ + {I}_{n} )}^{- 1} )}^{2} ( {σ}_{R}^{2} Σ + ϕ {I}_{n} ) ] \\ = ϕ tr [ {( {\tilde{σ}}_{R}^{2} Σ + {I}_{n} )}^{- 1} ] \\ = ϕ {\sum }_{i = 1}^{n} {( {\tilde{σ}}_{R}^{2} {λ}_{i} ( Σ ) + 1 )}^{- 1}\end{matrix} .$$

Proposition 2. Under the above notations, assume that σ²=ϕ and \tilde{σ}_{R}²≤\tilde{τ}min_{1≤l≤L}ξₗ, we have

APEKNN [symbol] APELMM,

where ALEKNN stands for the average prediction error for kernel neural network and the symbol “ [symbol] ” denotes asymptotically less than.
Proof. For the KNN, the average prediction error is given by:

$$\begin{matrix}APEKNN = 𝔼 [ {y}^{T} {A}^{2} y ] = 𝔼 [ 𝔼 ( {y}^{T} {A}^{2} y \vert {u}_{1} , … , {u}_{m} ) ] \\ = ϕ𝔼 [ tr ( {A}^{2} ( \tilde{τ} K ( U ) + {I}_{n} ) ) ] \\ = ϕ tr \{ {( 𝔼 [ {( \tilde{τ} K ( U ) + {I}_{n} )}^{- 1} ] )}^{2} 𝔼 [ \tilde{τ} K ( U ) + {I}_{n} ] \} \\ ≄ ϕ tr \{ {( \tilde{τ} {\sum }_{l = 1}^{L} {ξ}_{l} {K}_{l} ( X ) + {I}_{n} )}^{- 2} ( \tilde{τ} {\sum }_{l = 1}^{L} {ξ}_{l} {K}_{l} ( X ) + {I}_{u} ) \} \\ = ϕ tr \{ {( \tilde{τ} {\sum }_{l = 1}^{L} {ξ}_{l} {K}_{l} ( X ) + {I}_{n} )}^{- 1} \} \\ \leq ϕ {\sum }_{i = 1}^{n} {( \tilde{τ} {min}_{1 \leq l \leq L} {ξ}_{l} {λ}_{i} ( {\sum }_{l = 1}^{L} {K}_{l} ( X ) ) + 1 )}^{- 1}\end{matrix}$$

Under the assumptions in this proposition, we have for the linear mixed model with Σ=Σₗ₌₁^{L}Kₗ(X),

$$\frac{{ϕ ( \tilde{τ} {min}_{1 \leq l \leq L} {λ}_{i} ( Σ ) + 1 )}^{- 1}}{{{σ}^{2} ( {\tilde{σ}}_{R}^{2} {λ}_{i} ( Σ ) + 1 )}^{- 1}} = \frac{ϕ}{{σ}^{2}} \frac{{\tilde{σ}}_{R}^{2} {λ}_{i} ( Σ ) + 1}{\tilde{τ} {min}_{1 \leq l \leq L} {ξ}_{l} {λ}_{i} ( Σ ) + 1} \leq 1 ,$$

which implies tha APELNN [symbol] APELMM.

[symbol]

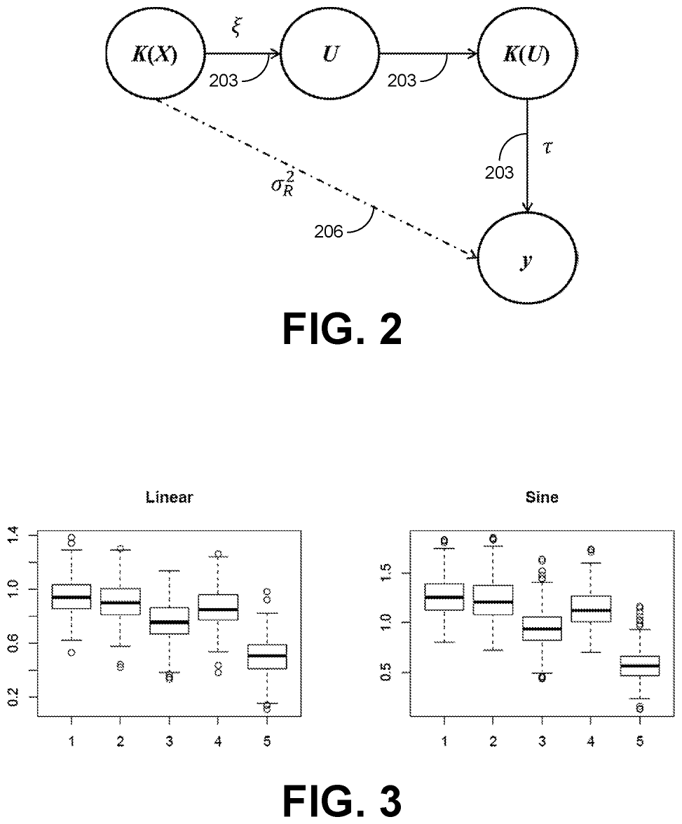

Remark 2. The assumption in Proposition 2 can be interpreted as follows: As shown in the FIG. 2 for the case of L=1, there are two paths from the kernel matrix based on X to the response y. One is the kernel neural network path (solid line 203) and the other is the linear mixed model path (dash-dotted line 206). The intuition behind the assumption \tilde{σ}_{R}²≤\tilde{τ}ξ is that for the kernel neural network, since it has two portions, it should explain more variations than the linear mixed model does.

The result may be extended to

$$K ( u ) = f [ \frac{1}{m} U {U}^{T} ] ,$$

where f is as described in Lemma 3(ii).

Proposition 3. Under the above notations, assume that σ²=ϕ, \tilde{σ}_{R}²≤\tilde{τ} min_{1≤l≤L}83 ₗ and λ₁((f−l)[Σₗ₌₁^{L}ξₗKₗ(X)])≥0with |f″(x)|≤M for some M>0 and all x between min_{i,j}σᵢⱼ and

$${max}_{i , j} \frac{{v}_{i}^{T} {v}_{j}}{m} ,$$

then one gets:

APEKNN [symbol] APELMM,

where λ₁(Σ) is the smallest eigenvalue of the matrix Σ.
Proof. Note that:

$$\begin{matrix}APEKNN = ϕ tr \{ {( 𝔼 [ {( \tilde{τ} K ( U ) + {I}_{n} )}^{- 1} ] )}^{2} 𝔼 [ \tilde{τ} K ( U ) + {I}_{n} ] \} \\ ≃ ϕ tr \{ {( \tilde{τ} f [ {\sum }_{l = 1}^{L} {ξ}_{l} {K}_{l} ( X ) ] + {I}_{n} )}^{- 1} \} \\ = ϕ {\sum }_{i = 1}^{n} \frac{1}{\tilde{τ} {λ}_{i} ( f [ {\sum }_{l = 1}^{L} {ξ}_{l} {K}_{l} ( X ) ] ) + 1} \\ \leq ϕ {\sum }_{i = 1}^{n} \frac{1}{\begin{matrix}\tilde{τ} {λ}_{i} ( ( f - ι ) [ {\sum }_{l = 1}^{L} {ξ}_{l} {K}_{l} ( X ) ] + \\ {min}_{1 \leq l \leq L} {ξ}_{l} {\sum }_{l = 1}^{L} {K}_{l} ( X ) ) + 1\end{matrix}}\end{matrix} ,$$

where l:Σ [symbol] Σ is the identity map. Corollary 4.3.15 in Matrix Analysis by R. A. Horn and C. R. Johnson (Cambridge University Press, 2nd ed., 2012) implies that:

$$ϕ {\sum }_{i = 1}^{n} \frac{1}{\tilde{τ} {min}_{1 \leq l \leq L} {ξ}_{l} {λ}_{i} ( {K}_{l} ( X ) ) + \tilde{τ} {λ}_{1} ( ( f - ι ) [ {\sum }_{l = 1}^{L} {ξ}_{l} {K}_{l} ( X ) ] ) + 1}$$

Corollary 1. If . f[Σₗ₌₁^{L}ξₗKₗ(X)]−Σₗ₌₁^{L}ξₗKₗ(X) is positive semidefinite, then:

APEKNN [symbol] APELMM,

Example 1 (Polynomial Kernels). For polynomial kernel of degree d, i.e.,

$${K}_{i j} ( U ) = {( c + \frac{{w}_{i}^{T} {w}_{j}}{m} )}^{d} ,$$

then

$$f ( x ) = {( c + x )}^{d} = {Σ}_{k = 0}^{d} ( \begin{matrix}d \\ k\end{matrix} ) {c}^{d - k} {x}^{k}$$

so that:

$$( f - ι ) ( x ) = {c}^{d} + ( d {c}^{d - 1} - 1 ) x + {\sum }_{k = 2}^{d} ( \begin{matrix}d \\ k\end{matrix} ) {c}^{d - k} {x}^{k} .$$

Theorem 4.1 in “Monotonicity for entrywise functions of matrices” by F. Hiai (Linear Algebra and its Applications 431 (8), pp. 1125-1146, 2009) states that for a real function on (−α, α), 0<α≤∞, it is Schur positive if and only if it is analytic and f^{(k)}(0)≥0 for all k≥0. For a real function f on (−α, α) and for n∈ [symbol] , it is Schur-positive of order n if f[A] is positive semidefinite for all positive semidefinite A∈ [symbol] ₙ( [symbol] ) with entries in (−α, α). Since f−l is a polynomial function so that it is clearly analytic and expanding f(x) using Taylor expansion around 0, one can obtain:

$$( \begin{matrix}d \\ k\end{matrix} ) {c}^{d - k} = \frac{{f}^{( k )} ( 0 )}{k !} ⇒ {f}^{( k )} ( 0 ) = \frac{d !}{( d - k ) !} {c}^{d - k} , k = 0 , … , d .$$

Hence:

$${( f - ι )}^{( k )} ( x ) = \{ \begin{matrix}{dc}^{d - 1} - 1 & if k = 1 \\ \frac{d !}{( d - k ) !} {c}^{d - k} & if k ∈ \{ 0 , 1 , … , d \} / \{ 1 \} \\ 0 & k \geq d + 1\end{matrix} .$$

Then, to make f−l Schur positive, one only needs to require

$$c \geq {}^{d - 1} \sqrt{\frac{1}{d}} \geq 0$$

so that the minimum eigenvalue condition of Proposition 3 holds.

Simulations. Simulation studies have been conducted to compare the prediction performance of KNN and LMM. The simulation results are based on 100 individuals with 500 Monte Carlo iterations.

Nonlinear Random Effect. The performances of both methods are examined under the situation of nonlinear random effects. Specifically, the following model was used to simulate the response:

$$y = f ( a ) + ϵ , a ∼ {𝒩}_{n} ( 0 , \frac{1}{p} {GG}^{T} ) , \qquad ( 11 )$$

where G is an n×p matrix containing the genetic information (SNP). In the simulation, four types of functions f are considered, which are linear (f(x)=x), sine (f(x)=sin(2 πx)), inverse logistic (f(x)=1/(1+e^{-x})) and polynomial function of order 2 (f(x)=x²). When applying KNN, set L=J=1 with K(X) chosen as either product kernel or polynomial kernel of order 2 and K(U) also chosen as either product kernel or polynomial kernel. The prediction errors of LMM and KNN are summarized via boxplot.

FIG. 3 demonstrates the results when f is chosen to be linear or sine function. The boxplots summarize the prediction performance of linear mixed models (LMM) and kernel neural networks (KNN) in terms of prediction errors. The left panel shows the results when a linear function is used, and the right panel shows the results when a sine function is used. In the horizontal axis, “1” corresponds to the LMM; “2” corresponds to the KNN with product input kernel and product output kernel; “3” corresponds to the KNN with product input and polynomial output; “4” corresponds to the KNN with polynomial input and product output and “5” corresponds to the polynomial input and polynomial output.

The results for all four cases are similar as shown from the boxplots. As expected, when the output kernel is chosen to be the product kernel, the performance of KNN is similar to the performance of LMM. Although when the input kernel is chosen to be a polynomial kernel, a slightly better prediction error is obtained. The situations change significantly when the output kernel is chosen to be the polynomial kernel. As one can tell from the box plots, when both the input and output kernels are chosen to be polynomial, the KNN has the best performance in terms of the prediction error. Such a pattern is consistent in all the simulations conducted in this section.

Nonadditive Effects. The performances of both methods are evaluated under nonadditive effects. Two simulations were conducted in terms of two different types of nonadditive effects. In the first simulation, we focus on the interaction effect and generate the response using the following model:

y=f(G)+ϵ,

where G=[g₁, . . . , gₚ] is the SNP matrix and ϵ˜ [symbol] (0, Iₙ). When applying both methods, the mean is adjusted so that the response has a marginal mean of 0. In the simulation, 10 causal SNPs denoted by gᵢ_{₁}, . . . , gᵢ_{₁₀} were randomly picked and:

$$f ( G ) = {\sum }_{1 \leq {j}_{1} < {j}_{2} \leq 10} {g}_{{i}_{{j}_{1}}} ⊙ {g}_{{i}_{{j}_{2}}} .$$

where ⊙ stands for the Hadamard product. When LMM was applied in the simulation, the product kernel was used as the covariance matrix for the random effect.

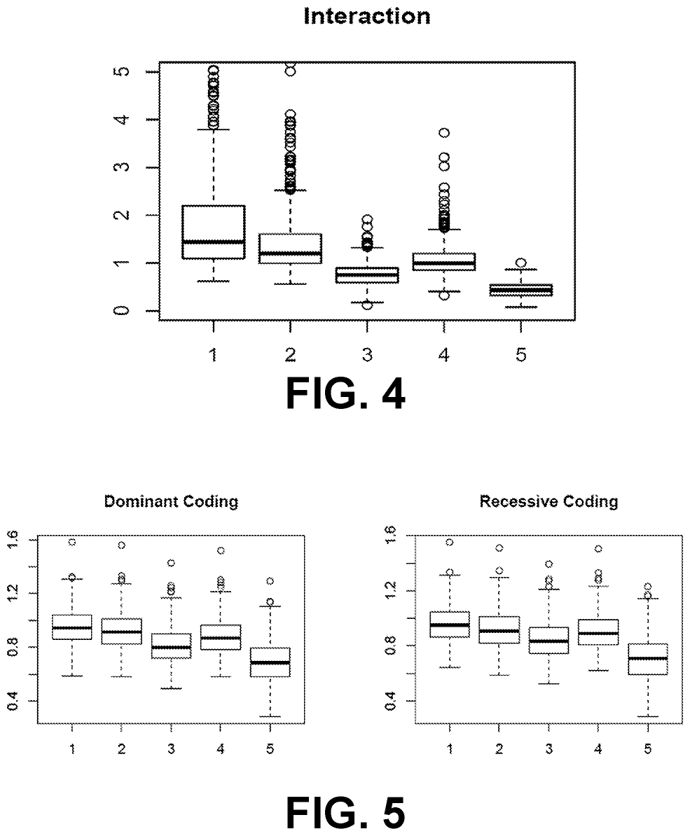

The result of LMM and KNN in the presence of interaction effect is shown in FIG. 4. The boxplots summarize the prediction errors of LMM and KNN. The vertical axis is scaled to 0-5 by removing some outliers to make the comparison visually clear. In the horizontal axis, “1” corresponds to the LMM; “2” corresponds to the KNN with product input kernel and product output kernel; “3” corresponds to the KNN with product input and polynomial output; “4” corresponds to the KNN with polynomial input and product output and “5” corresponds to the polynomial input and polynomial output. It is interesting to see from the boxplots that both LMM and KNN has many outliers when the product kernel is applied. Overall, LMM has larger variations compared to KNN in this scenario. When the output kernel in KNN is specified as the polynomial kernel, the performance of KNN is much better than that of LMM.

There are three main coding schemes for single-nucleotide polymorphism (SNP): additive coding, dominant coding and recessive coding. In many situations, the additive coding (AA=0, Aa=1, aa=2) is used. In the second simulation, consider the scenarios when the dominant coding (AA=1, Aa=1, aa=0) and recessive coding (AA=0, Aa=1, aa=1) are used for SNP data. The response was simulated based on the model:

$$y = a + ϵ , a ∼ {𝒩}_{n} ( 0 , \frac{1}{p} {G}^{′} {G}^{′ T} ) , ϵ ∼ {𝒩}_{n} ( 0 , {I}_{n} ) ,$$

where G′ is a SNP matrix either using dominant coding or recessive coding so that each element in G′ takes only two possible values 0 and 1.

FIG. 5 summarizes the simulation results of LMM and KNN in terms of prediction errors when dominant coding (top figure) and recessive coding (bottom figure) are used. In the horizontal axis, “1” corresponds to the LMM; “2” corresponds to the KNN with product input kernel and product output kernel; “3” corresponds to the KNN with product input and polynomial output; “4” corresponds to the KNN with polynomial input and product output and “5” corresponds to the polynomial input and polynomial output. By comparing the two boxplots in FIG. 5, the performances look similar in both cases. As in the other case, KNN with polynomial kernel input and polynomial kernel output achieves the lowest prediction error.

Real Data Application. The disclosed method was applied to the whole genome sequencing data from Alzheimer's Disease Neuroimaging Initiative (ADNI). A total of 808 individuals at the baseline of the ADNI1 and ADNI2 studies have the whole genome sequencing data. We dropped the single nucleotide polymorphisms (SNPs) with low calling rate (<0.9), or low minor allele frequencies (MAF) (<0.01), or those failed to pass the Hardy Weinberg exact test (p-value<1e−6), and non-European American samples were also dropped. The data was then uploaded to the server in the University of Michigan for posterior likelihood imputation (https://imputationserver.sph.umich.edu/index.html). From the imputed data, we extracted SNPs with allelic R²>0.9 and then the covariance kernel matrix, genetic relationship kernel matrix used in “Gcta: a tool for genome-wide complex trait analysis” by Yang et al. (The American Journal of Human Genetics 88 (1), pp. 76-82, 2011) and the normalized identity-by-state (IBS) kernel matrix were constructed for analysis. Specifically, the (i,j)th element in each of the three kernel matrices is defined as follows:

$${K}^{cov} ( {g}_{i} , {g}_{j} ) = \frac{1}{p - 1} {\sum }_{k = 1}^{p} ( {g}_{ik} - \frac{1}{p} {\sum }_{k} {g}_{ik} ) ( {g}_{jk} - \frac{1}{p} {\sum }_{k} {g}_{jk} )$$

$${K}^{rel} ( {g}_{i} , {g}_{j} ) = \frac{1}{p} {\sum }_{k = 1}^{p} \frac{( {g}_{ik} - 2 {p}_{k} ) ( {g}_{jk} - 2 {p}_{k} )}{2 {p}_{k} ( 1 - {p}_{k} )}$$

$${K}^{ibs} ( {g}_{i} , {g}_{j} ) = \frac{1}{2 p} {\sum }_{k = 1}^{p} [ 2 - \vert {g}_{ik} - {g}_{jk} \vert ] .$$

where p is the number of SNPs in all expressions. In K^{rel}(gᵢ, gⱼ) is the frequency of the reference allele.

Four volume measures of cortical regions, which are hippocampus, ventricles, entorhinal and whole brain volumes were used as phenotypes of interest. These four cortical regions were chosen since they play important roles in Alzheimer's disease (AD). The loss in the volumes of the whole brain, hippocampus and entorhinal and the increment in the ventricular volume can be detected among AD patients. When both KNN and LMM were applied, only the subjects having both genetic information and phenotypic information were included, which resulted in 513 individuals for hippocampus; 564 individuals for ventricles; 516 individuals for entorhinal and 570 individuals for whole brain volumes.

First, a simple linear regression with the response variable being the natural logarithm of the volumes of the four cortical regions was performed. The covariates were chosen as the age, gender, education status and APOE4. After that, the residuals e were extracted as the new response variable for the KNN and LMM methods. Specifically, the LMM is based on the following model assumption:

e˜( [symbol] (0, τ₁K^{cov}+τ₂K^{rel}+τ₃K^{lbs}+Σ₄Iₙ). (12)

The maximum likelihood estimates for τᵢ, i=1,2,3,4 were then calculated based on the Fisher scoring methods and the BLUP for the LMM were computed based on these estimators. Similarly, when we applied the KNN methods, the three kernel matrices K^{cov}, K^{rel} and K^{lbs} were used as the input kernel matrices and the output kernel matrix is chosen to be either the product kernel or the polynomial kernel of order 2. The average mean square errors on predictors of both methods were summarized in Table 1, which shows the average mean squared prediction error of KNN with product output kernel matrix, KNN with output kernel matrix as the polynomial of order 2, and the BLUP based on LMM.

TABLE 1

| | KNN(prod) | KNN(poly) | LMM |
|---|---|---|---|
| Hippocampus | 2.44e−03 | 8.50e−07 | 8.60e−03 |
| Ventricles | 2.49e−02 | 2.78e−05 | 1.17e−01 |
| Entorhinal | 3.06e−03 | 1.56e−06 | 5.71e−15 |
| Whole Brain Volume | 6.31e−04 | 7.94e−07 | 5.48e−04 |

As can be seen from the table, in most scenarios, the KNN with the polynomial output kernel matrix has lower prediction error, which agrees with the empirical results we obtained from the simulation studies. It is interesting to note that the average prediction error of LMM for entorhinal is extremely close to zero. We checked the estimates of the variance components in this case and noticed that the variance component is associated with the identity matrix, which is τ₄ as in equation (12). Since the BLUP of the random effect in this case is given by:

BLUP=(τ₁K^{cov}+τ₂K^{rel}+τ₃K^{lbs})(τ₁K^{cov}+τ₂K^{rel}+τ₃K^{lbs}+Σ₄Iₙ)⁻¹e

so that if τ₄ is very close to 0, it can be known that the BLUP is very close to the response e, which leads to the extremely low prediction error.

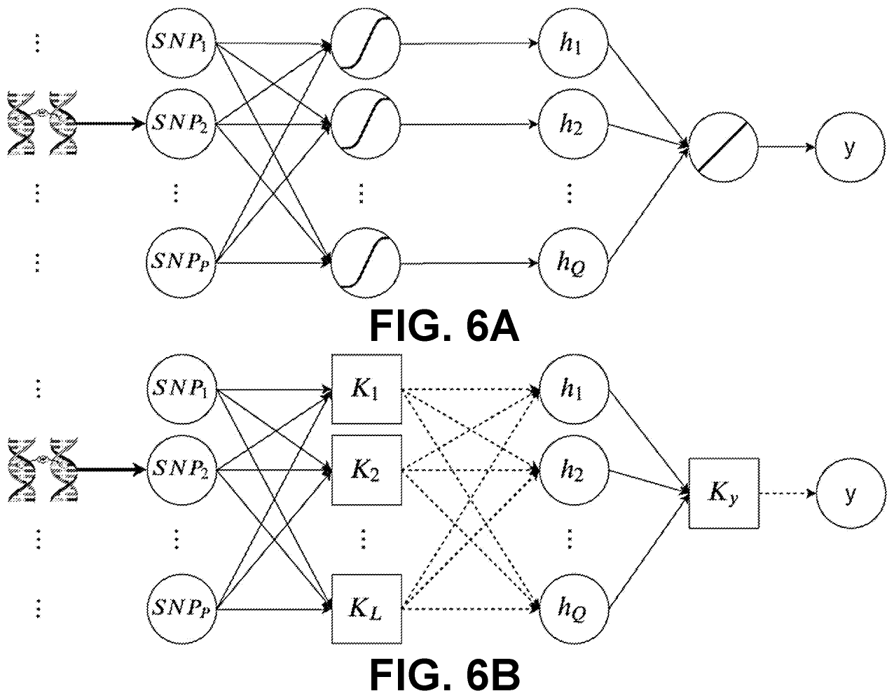

KNN inherits features from both LMM and classical neural networks. KNN has a similar network structure as classical neural networks but uses kernel matrix as inputs. FIG. 6 illustrates side-by-side network structures of a classical neural network and KNN. KNN can also be thought of as an extension of LMM since it can reduce to LMM through choosing product kernel matrix as the output kernel matrix and via reparameterization. Empirical simulation studies and real data applications show that the KNN model can achieve better performance than classic methods.

While KNN has many advantages, fitting KNN on large-scale genomic datasets (e.g., half a million samples) could also bring computational challenges. For studies with a large number of samples (e.g., half a million), we introduce a batch training approach to speed up the computation. In the batch training process, we randomly partition the study sample into M equal-sized batches. KNN is applied to each batch, resulting in M random effect estimates. We can then average these M estimates to obtain the estimates for the random effects. More specifically, we randomly partition the training cohort [symbol] ={y,X,Z} into M equally-sized batches { [symbol] ₁, . . . , [symbol] _{M}} and fit KNN on each batch, resulting in M sets of estimates \hat{θ}={\hat{θ}₁, . . . , \hat{θ}_{M}}. Then, taking the average as the final estimate:

$$\hat{θ} = \frac{1}{M} {\sum }_{m = 1}^{M} {\hat{θ}}_{m} , {\hat{θ}}_{m} = {arg min}_{θ} loss ( {𝕐}_{m} ; θ ) . \qquad ( 23 )$$

Treating [symbol] as the population, and [symbol] ₁, . . . , [symbol] _{M} as random independent samples from this population, the parameter θₘ associated with batch m will be an unbiased estimate of the actual parameter θ. By the rule of large numbers, the averaged

$$\overline{θ} = \frac{1}{M} {\sum }_{m = 1}^{M} {θ}_{m}$$

will converge to θ in probability when the number of batches M goes to infinity (with fixed batch size, implying that the total sample size also approaches infinity). In practice, each batch model θₘ is replaced with the estimated \hat{θ}ₘ, and the average θ̅ is replaced with \hat{θ} (equation 23). The computation time will then be reduced from O(N³) to

$$O ( \frac{{N}^{3}}{{M}^{2}} ) .$$

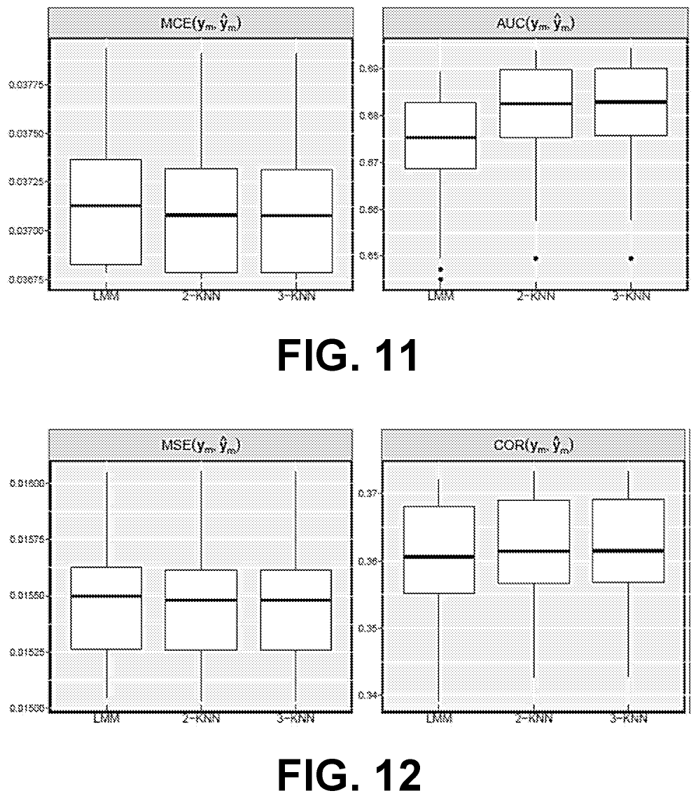

his batched training is illustrated in FIG. 11.

To further speed up the training of each batch model, a minimum norm quadratic unbiased estimation (MINQUE) can be used as the variance component solver. Unlike widely-used REML that requires iterations, MINQUE has the appealing feature of giving unbiased solutions in a closed-form. Though less consistent than REML, the ability to handle massive sample sizes can offset such drawbacks.

To evaluate the performance of the α-KNN method trained with data [symbol] ={y_{C},X_{C},Z_{C}}, compare the predicted ŷ_{B} with the actual y_{B} in a testing dataset [symbol] ={y_{B},X_{B},Z_{B}}. A seemingly quick solution would be to apply the estimated \hat{θ} to the widely used best linear unbiased predictor (BLUP) to make prediction ŷ_{B}. In this case, however, BLUP is invalid because ŷ_{B} can be made arbitrarily close to y_{B} by merely underestimating the noise variance σ₀². Instead, a “leave one out predictor” (LOOP) can be used to produce ŷ_{B}.

Simulation Results. Additional simulations with a large number of variants and samples were conducted based on imputed genotypes on chromosome 20. Out of 2,082,571 variants on chromosome 20, those with (a) imputation information score no smaller than 0.3 (on a 0 to 1 scale), (b) minor allele frequency (MAF) no less than 0.01, (c) missing-rate no more than 2%, and (d) a p-value of Hardy-Weinberg equilibrium (HWE) test no less than 1×10⁻⁶ were kept. After the quality control procedure, 125,515 variants remained for the simulations. Furthermore, the simulations were restricted to 352,962 unrelated individuals of White-British descent, with a missing rate of no more than 2%. Additional exclusion criteria included outliers of excess heterozygosity, sex information mismatched with genetically inferred sex, and withdrawal of informed consent.

Next, N=2,000 individuals and P=4,000 variants from a chromosome region were randomly selected for training, and another 2,000 individuals with the same set of variants were randomly selected for testing, resulting in the typical P>N scenario for human genetic studies. To generate the outcome y from an N×P genotype matrix Z, the LMM model (equations 13 and 17) was followed. Specifically, a small proportion (e.g., γ=5%) of variants were randomly selected to form casual genotype sub-matrix, \tilde{Z}. The causal genetic effects then follow

$$g ∼ 𝒩 ( 0 , \frac{{σ}_{g}^{2}}{P γ} \tilde{Z} {\tilde{Z}}^{′} ) .$$

The simulated outcome was thus y=g+ϵ, where ϵ˜ [symbol] (0, σ₀²I). To evaluate the impact of epistasis, variant interactions (i.e., specific epistasis) or nonlinear genetic effects such as hyperbola and Ricker-curve growth functions (i.e., non-specific epistasis) were simulated. A heavy-tailed Student-t random error was further introduced to evaluate the robustness of the proposed method. A list of simulated scenarios includes:
α-way interactions: expand the genotype matrix Z by adding element-wise products of α variants as new columns.
Nonlinear genetic effects:

$$α - power : \tilde{g} = {g}^{α} \qquad ( 24 )$$

$$α - hyperbola : \tilde{g} = \frac{r ( {g}^{α} )}{1 + r ( {g}^{α} )} \qquad ( 25 )$$

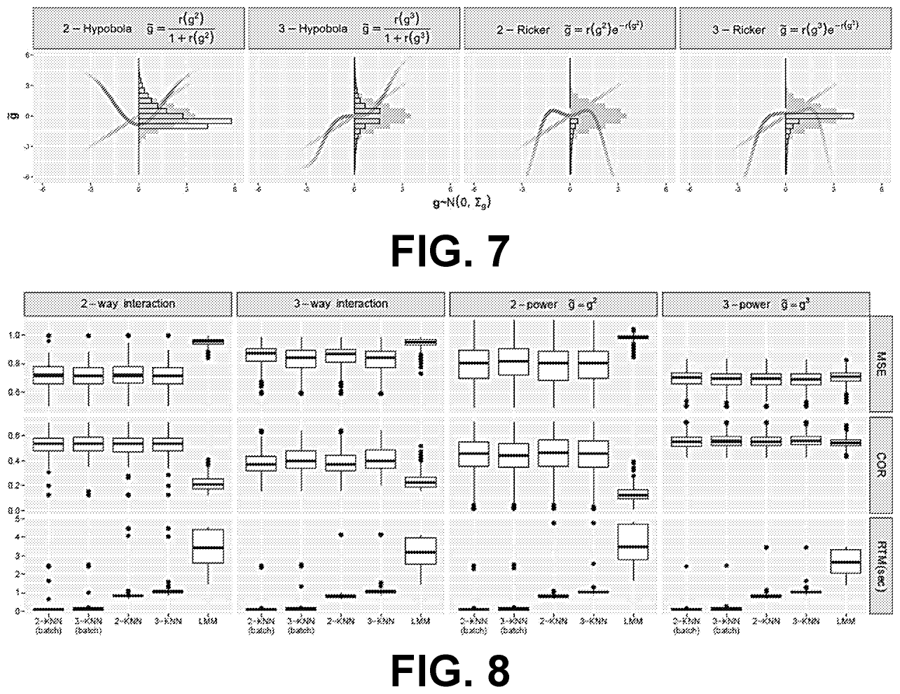

α−Rickercurve: \tilde{g}=r(g^{α})exp[−r(g^{α})], (26)

where r is the soft rectifier, r(x)=log(1+e^{x}), that transforms raw genetic effects into non-negative stimuli required by a growth function. The two types of growth functions are illustrated in FIG. 7—hyperbola and Ricker. The x-axis is the original genetic effects g˜ [symbol] (0, Σ_{g}) and the y-axis is the nonlinear genetic effects \tilde{g}. The shaded portions indicate values and distribution of unchanged genetic efforts and the bars indicated valued and distribution of nonlinear genetic effects.
Student-t-noise:

$${\tilde{ϵ}}_{i} {∼}^{i . i . d .} t ( \frac{2 {σ}_{0}^{2}}{{σ}_{0}^{2} - 1} ) . \qquad ( 27 )$$

where the degrees of freedom were chosen such that var(\tilde{ϵ})=var(ϵ)=σ₀².

For model comparison, evaluation was carried out for the mean square error (MSE), and correlation (COR) between truth and predictions made by (1) LMM trained by REML implemented in GCTA, (2) KNN trained by MINQUE, and (3) the same KNN trained on four batches of 500 each. We also compared the running time (RTM) for training each model. All five models used the same product kernel (equation 18) built from the same genotype. Each simulation scenario was repeated 500 times.

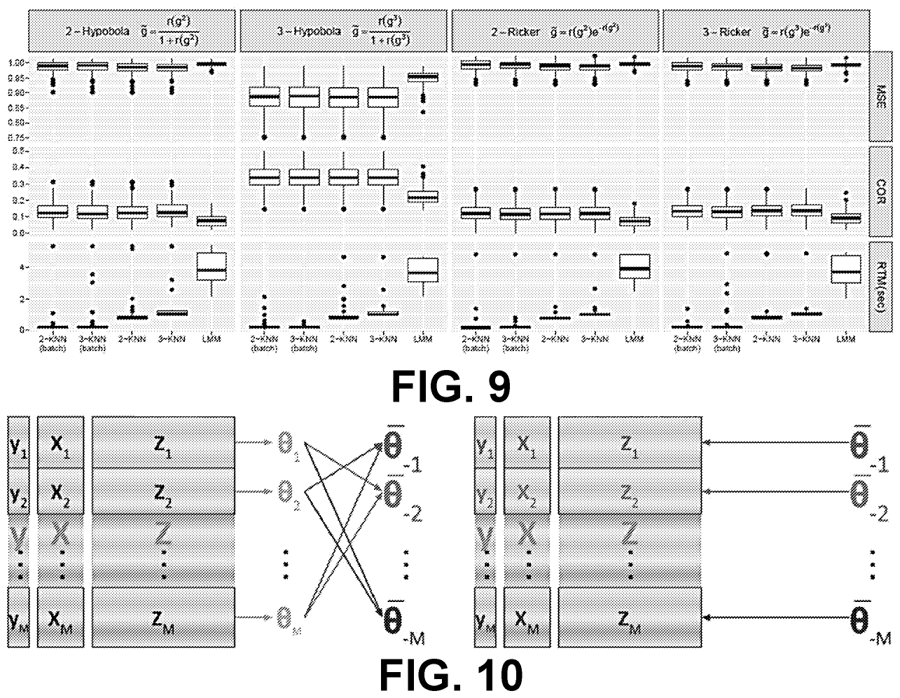

FIGS. 8 and 9 summarize simulation results and show that KNN produced significantly more accurate predictions than LMM when epistasis was driven by interactions (i.e., specific epistasis, FIG. 8, Columns 1 and 2), which is expected because LMM only captures additive effects and ignores interactions. In FIG. 8 (left to right), epistasis is driven 2-way interactions (column 1), 3-way interactions (column 2), quadratic genetic effect (column 3), and cubic genetic effect (column 4); and (top to bottom), prediction accuracy measured by mean square error (MSE, row 1), correlation (COR, row 2), and the running time to train a model in seconds (RTM, row 3). FIG. 9 shows epistasis due to nonlinear growth functions with (left to right) 2nd order hyperbola (column 1), 3rd order hyperbola (column 2), 2nd order Ricker curve (column 3), and 3rd order Ricker curve (column 4); and (top to bottom), prediction accuracy measured by mean square error (MSE, row 1), correlation (COR, row 2), and the running time to train a model in seconds (RTM, row 3). The competing modeling strategies include KNN trained by batched MINQUE, KNN trained by MINQUE with the undivided sample, and LMM trained by REML with the undivided sample.

With some nuances, for the three-way interactions, KNN with 2-order polynomial output kernel (2-KNN) was slightly less accurate than KNN with 3-order polynomial output kernel (3-KNN), suggesting the lack of fit due to model under-specification. For the two-way interactions, though, 3-KNN was as precise as correctly specified 2-KNN, indicating the robustness of KNN against model over-specification. When epistasis was due to a nonlinear genetic effect on phenotype (i.e., non-specific epistasis), KNN outperformed LMM by a noticeable margin in 5 out of 6 cases, (FIG. 8 Column 3, and FIG. 9 Columns 1-4), except for the cubic power function (FIG. 8 Column 4), where KNN outperforms LMM by a relatively small margin. Additionally, FIGS. 8 and 9 illustrate that batch-trained KNN s are less accurate than their counterpart trained on the entire sample but by a margin small enough to retain the advantages over LMM in all scenarios considered.

The running times of training are illustrated in the third row of FIGS. 8 and 9 show the computational efficiency of the proposed method. In all scenarios, the required times to train a model, ordered from the longest to the shortest, were LMM (by REML)>>3-KNN>2-KNN>>3-KNN (by batch)>2-KNN (by batch). This descending order of running times demonstrates the intended reduction of computation time by our method, achieved by replacing REML with MINQUE to avoid iterations, and by dividing the total sample into M batches to further downscale the computation by a factor of M².

Analysis of UK Biobank Phenotypes. The method can be showcased by applying it to the UK Biobank data comprised of 805,426 variants on 488,377 individuals, 43 health-related phenotypes (listed in Table 2), and covariates. Variants with a missing rate no more than 0.02, MAF no less than 0.01, and HWE p-value no less than 1×10⁻⁶ were retained. The samples of unrelated White-British descent with a missing rate of no more than 0.01 were also retained. Additional exclusion criteria, once again, included outliers of excess heterozygosity, sex mismatches with genetically inferred sex, and withdrawal of informed consent. This quality control procedure left a working sample of N=341,196 individuals and P=405,455 autosome variants.

TABLE 2
UKB Phenotypes

| name | type | N(case/non-zero %, male %) | age(S.D) |
|---|---|---|---|
| allergy | binary | 375,649(7.5%, 45.9%) | 56.7(8.0) |
| alcohol per week | numeric | 374,597(77.0%, 45.9%) | 56.7(8.0) |
| asthma | binary | 375,649(11.5%, 45.9%) | 56.7(8.0) |
| back problems | binary | 375,649(5.9%, 45.9%) | 56.7(8.0) |
| bipolar/major depression | binary | 90,869(27,7%, 46.9%) | 57.2(8.0) |
| bowel problems | binary | 375,649(6.7%, 45.9%) | 56.7(8.0) |
| melanoma and skin | binary | 375,649(4.5%, 45.9%) | 56.7(8.0) |
| skin cancer | binary | 375,649(3.9%, 45.9%) | 56.7(8.0) |
| breast cancer | binary | 203,247(4.9%, 0.0%) | 56.5(7.9) |
| prostate cancer | binary | 172,402(3.7%, 100.0%) | 56.9(8.1) |
| any cancer | binary | 375,649(14.9%, 45.9%) | 56.7(8.0) |
| cardiovascular | binary | 375,649(36.3%, 45.9%) | 56.7(8.0) |
| maximum cannabis frequency | numeric | 122,490(21.9%, 43.6%) | 56.0(7.7) |
| In situ neoplasms | binary | 375,649(2.2%, 45.9%) | 56.7(8.0) |
| diastolic blood pressure | numeric | 346.066(100.0%, 46.0%) | 56.7(8.0) |
| depression | binary | 375.649(5.8%, 45.9%) | 56.7(8.0) |
| endocrine/diabetes | binary | 375,649(10.3%, 45.9%) | 56.7(8.0) |
| fluid intelligence score | numeric | 122,697(99.9%, 45.9%) | 57.0(8.1) |
| gynaecology/breast | binary | 375,649(5.4%, 45.9%) | 56.7(8.0) |
| gastrointestinal/abdominal | binary | 375,649(16.2%, 45.9%) | 56.7(8.0) |
| hayfever/allergic rhinitis | binary | 375,649(5.7%, 45.9%) | 56.7(8.0) |
| haematology/dermatology | binary | 375,649(5.5%, 45.9%) | 56.7(8.0) |
| hyper-cholesterol | binary | 375,649(12.1%, 45.9%) | 56.7(8.0) |
| hypertension | binary | 375,649(26.4%, 45.9%) | 56.7(8.0) |
| heart problems | binary | 375,649(7.1%, 45.9%) | 56.7(8.0) |
| any illness | binary | 375,649(74.1%, 45.9%) | 56.7(8.0) |
| count illness | numeric | 375,649(74.1%, 45.9%) | 56.7(8.0) |
| immuno/systemic disorders | binary | 375,649(8.7%, 45.9%) | 56.7(8.0) |
| joint disorder | binary | 375,649(12.5%, 45.9%) | 56.7(8.0) |
| musculoskeletal/trauma | binary | 375,649(22.0%, 45.9%) | 56.7(8.0) |
| mean time to memorize | numeric | 373,105(100.0%, 45.9%) | 56.7(8.0) |
| neurology/eye/psychiatry | binary | 375,649(17.3%, 45.9%) | 56.7(8.0) |
| neurological | binary | 375,649(6.8%, 45.9%) | 56.7(8.0) |
| neuroticism | numeric | 304,432(84.7%, 46.7%) | 56.5(8.0) |
| oesophageal disorder | binary | 375,649(6.5%, 45.9%) | 56.7(8.0) |
| osteoarthritis | binary | 375,649(8.1%, 45.9%) | 56.7(8.0) |
| prospective memory | binary | 125,524(20.9%, 46.0%) | 57.0(8.1) |
| psychological/psychiatric | binary | 375,649(7.6%, 45.9%) | 56.7(8.0) |
| respiratory | binary | 375,649(17.9%, 45.9%) | 56.7(8.0) |
| systolic blood pressure | numeric | 346,062(100.0%, 46.0%) | 56.7(8.0) |
| pack/year of smoking | numeric | 374,352(30.3%, 45.9%) | 56.7(8.0) |
| thyroid problem | binary | 375,649(5.8%, 45.9%) | 56.7(8.0) |

Next, the entire sample was divided into 170 batches of around 2,000 individuals each, using PLINK1.9. It was ensured that the distribution of the phenotype and covariates in each batch was consistent with the overall data. The product kernels (i.e., GRM) were then calculated for all the batches, again using PLINK1.9. For each batch, 2-KNN and 3-KNN were trained with MINQUE, as well as LMM with REML, for reference. Age, sex (except for prostate and breast cancer), the first four genetic principle components, and the genotyping phase, were included as fixed effects. The mean times to train a batch of about 2,000 individuals, for different models, are shown in Table 3.

TABLE 3
Training time (in seconds) for a batch of 2000 individuals.

| Model | Trained by | Training time (SD) |
|---|---|---|
| LMM | REML | 6.41(5.56) |
| 2-KNN | MINQUE | 1.96(1.57) |
| 3-KNN | MINQUE | 2.12(1.74) |

To evaluate prediction performance, exploit the fact that cohort [symbol] ={y, X, Z} has divided into M parts { [symbol] ₁, . . . [symbol] _{M}}, and the target model is an aggregation of batch models in Θ={θ₁, . . . , θ_{M}}. Therefore, some batches can be kept as testing data while the remaining used to train the model. In particular, let the M batches take turns to be the testing data one at a time, and use the rest M batches to train the model and estimate the parameters:

$${\overline{θ}}_{- m} = \frac{1}{M - 1} {\sum }_{\tilde{m} \neq m} {θ}_{\tilde{m}} , m = 1 ... M , \qquad ( 28 )$$

where the mth batch [symbol] ₘ={yₘ,Xₘ,Zₘ} was not a part of θ̅₋ₘ's training. Therefore, testing the model θ̅₋ₘ on the mth batch [symbol] ₘ was valid. This evaluation process constitutes the “leave one batch out” (LOBO) testing, where no part of the cohort was kept out of training. An example of the LOBO procedure is illustrated in FIG. 10. In the left panel, M models θ̅₁, . . . , θ̅_{M} were trained on M batches, which aggregated into M leave one batch out (LOBO) models θ̅₋₁, . . . ,θ̅₋ₘ. In the right panel, the mth LOBO model θ̅₋ₘ can be tested on the mth batch since that part of the data does not contribute to θ̅₋ₘ resulting in M tests.

For a particular LOBO model, θ̅₋ₘ, and its corresponding testing data [symbol] ₘ={yₘ,Xₘ,Zₘ}, prediction accuracy was measured by (1) Mean Classification Error (MCE) and the Area Under the Curve (AUC) for binary phenotypes, or (2) Mean Square Error (MSE) and correlation (COR) for continuous phenotypes. Prediction performances for one binary phenotype (skin cancer) and one continuous phenotype (systolic blood pressure) are shown in FIGS. 11 and 12, respectively. FIGS. 11 and 12 illustrate the box-plot of LOBO prediction performance measured on 170 batches, by mean square error (MSE) and correlation (COR) between observed and predicted disease with LMM and KNN.

Prediction performances for the remaining 41 phenotypes were evaluated. In general, for 42 out of 43 phenotypes (except mean time to memorize), both 2-KNN and 3-KNN predicted more accurately than LMM. Moreover, 3-KNN performed slightly better than 2-KNN for the majority of phenotypes. These results suggest that KNN managed to capture epistasis due to 2-way interactions, or 2-order nonlinear genetic effects missed by an additive model (i.e., LMM). Furthermore, it seems epistasis of higher-order may exist but is either too weak to show a significant impact or too challenging to capture with 3-KNN. Finally, note that the advantage of KNN was more apparent for binary phenotypes than for continuous ones, suggesting that genetic effects are still predominantly additive for continuous phenotypes.

Modeling genome-wide epistasis with kernels for improved predictions has long been suggested, however despite the rich material base provided by large cohorts like UK Biobank, the inhibiting cost of training a sophisticated model on a large sample has kept epistasis out of the mainstream research of human genetics.

Here, an M-batched KNN, trained with MINQUE, is proposed that is significantly faster than the most popular REML implementation. As a result, KNN can analyze large cohorts, such as the UK Biobank. It also provides superior prediction, demonstrated by both simulated and real data analysis. A significant improvement was seen in prediction accuracy of 2-KNN and 3-KNN over LMM, suggesting that epistasis does exist, and KNN can capture it.

In this study, a single GRM was used for the entire genome. To apply KNN more effectively, one may allow multiple kernels to model variants of several classes according to prior knowledge, such as location (e.g., chromosomes), function (e.g., coding, non-coding, or intergenic) or through supervised learning. Aside from greater flexibility, a multitude of kernels built from subgroups of variants have better diagonal/off-diagonal balance than a single, whole-genome kernel. To be noted, the gain of flexibility may reduce model stability, and increase computation time by a factor of

$$O ( ( \begin{matrix}L \\ α\end{matrix} ) )$$

when using L kernels. Growth in cohort size and computation power may offset these difficulties. Another way to improve the performance KNN is to select influential variants based on a significance criterion, such as GWAS p-values or SNP weights calculated by PRS-based approaches (e.g., LDPred). The selection may reduce the noise ratio among the remaining variants and improve the diagonal/off-diagonal balance in the GRM due to a reduced number of variants.

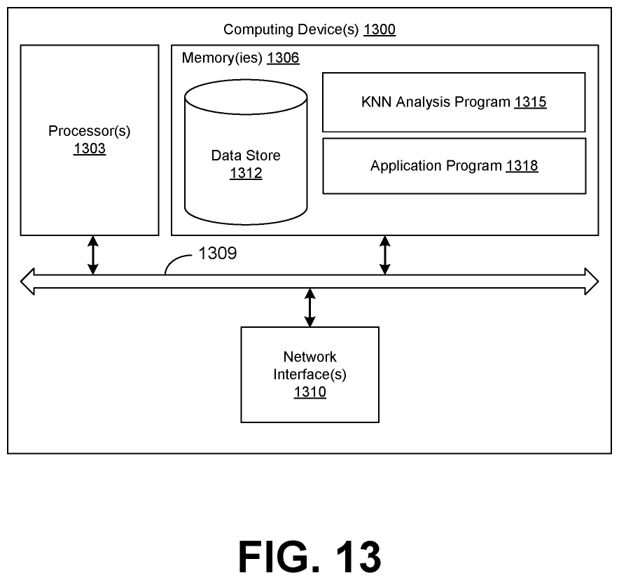

With reference to FIG. 13, shown is a schematic block diagram of a computing device 1300 that can be utilized to analyze patient data for diagnosis and/or recommend treatment or prevention using the KNN techniques. In some embodiments, among others, the computing device 1300 may represent a mobile device (e.g., a smartphone, tablet, computer, etc.). Each computing device 1300 includes at least one processor circuit, for example, having a processor 1303 and a memory 1306, both of which are coupled to a local interface 1309. To this end, each computing device 1300 may comprise, for example, at least one server computer or like device. The local interface 1309 may comprise, for example, a data bus with an accompanying address/control bus or other bus structure as can be appreciated.

In some embodiments, the computing device 1300 can include one or more network interfaces 1310. The network interface 1310 may comprise, for example, a wireless transmitter, a wireless transceiver, and a wireless receiver. As discussed above, the network interface 1310 can communicate to a remote computing device using a Bluetooth protocol. As one skilled in the art can appreciate, other wireless protocols may be used in the various embodiments of the present disclosure.

Stored in the memory 1306 are both data and several components that are executable by the processor 1303. In particular, stored in the memory 1306 and executable by the processor 1303 are a KNN analysis program 1315, application program 1318, and potentially other applications. Also stored in the memory 1306 may be a data store 1312 and other data. In addition, an operating system may be stored in the memory 1306 and executable by the processor 1303.

It is understood that there may be other applications that are stored in the memory 1306 and are executable by the processor 1303 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1306 and are executable by the processor 1303. In this respect, the term “executable” means a program file that is in a form that can ultimately be run by the processor 1303. Examples of executable programs may be, for example, a compiled program that can be translated into machine code in a format that can be loaded into a random access portion of the memory 1306 and run by the processor 1303, source code that may be expressed in proper format such as object code that is capable of being loaded into a random access portion of the memory 1306 and executed by the processor 1303, or source code that may be interpreted by another executable program to generate instructions in a random access portion of the memory 1306 to be executed by the processor 1303, etc. An executable program may be stored in any portion or component of the memory 1306 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1306 is defined herein as including both volatile and nonvolatile memory and data storage components. Volatile components are those that do not retain data values upon loss of power. Nonvolatile components are those that retain data upon a loss of power. Thus, the memory 1306 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1303 may represent multiple processors 1303 and/or multiple processor cores and the memory 1306 may represent multiple memories 1306 that operate in parallel processing circuits, respectively. In such a case, the local interface 1309 may be an appropriate network that facilitates communication between any two of the multiple processors 1303, between any processor 1303 and any of the memories 1306, or between any two of the memories 1306, etc. The local interface 1309 may comprise additional systems designed to coordinate this communication, including, for example, performing load balancing. The processor 1303 may be of electrical or of some other available construction.

Although the KNN analysis program 1315 and the application program 1318, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the KNN analysis program 1315 and the application program 1318, that comprises software or code can be embodied in any non-transitory computer-readable medium for use by or in connection with an instruction execution system such as, for example, a processor 1303 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the KNN analysis program 1315 and the application program 1318, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 1300, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method for risk prediction using high-dimensional and ultrahigh-dimensional data, comprising:
training a kernel-based neural network (KNN) with a training set of data to produce a trained KNN model, the KNN model comprising a plurality of kernels as a plurality of layers to capture complexity between the data with disease phenotypes, the training set of data comprising genetic information applied as inputs to the KNN and one or more phenotypes;
determining a likelihood of a condition based at least in part upon an output indication of the trained KNN corresponding to the one or more phenotypes, the output indication based upon analysis of data comprising genetic information from an individual by the trained KNN; and
identifying a treatment or prevention strategy for the individual based at least in part upon the likelihood of the condition.

2. The method of claim 1, wherein a first layer of the plurality of layers comprises a plurality of kernels and a last layer of the plurality of layers comprises a single kernel or a plurality of kernels.

3. The method of claim 2, wherein the plurality of kernels in the first layer converts a plurality of data inputs into a plurality of latent variants.

4. The method of claim 3, wherein the plurality of data inputs comprise single-nucleotide polymorphisms (SNPs) or biomarkers.

5. The method of claim 2, wherein individual latent variables of the plurality of kernels are generated by random sampling of outputs of the plurality of kernels.

6. The method of claim 2, wherein the single kernel or plurality of kernels of the last layer determines the output indication based upon a plurality of latent variable produced by a preceding layer of the plurality of layers.

7. method of claim 6, wherein the preceding layer is the first layer.

8. The method of claim 1, wherein the KNN is trained using minimum norm quadratic estimation.

9. The method of claim 1, wherein training of the KNN is accelerated using batch training.

10. A system for risk prediction, comprising:
at least one computing device comprising processing circuitry including a processor and memory, the at least one computing device configured to at least:
train a kernel-based neural network (KNN) with a training set of data to produce a trained KNN model, the KNN model comprising a plurality of kernels as a plurality of layers to capture complexity between the data with disease phenotypes, the training set of data comprising genetic information applied as inputs to the KNN and one or more phenotypes;
determine a likelihood of a condition based at least in part upon an output indication of the trained KNN corresponding to the one or more phenotypes, the output indication based upon analysis of data comprising genetic information from an individual by the trained KNN; and
identify a treatment or prevention strategy for the individual based at least in part upon the likelihood of the condition.

11. The system of claim 10, wherein a first layer of the plurality of layers comprises a plurality of kernels and a last layer of the plurality of layers comprises a single kernel or a plurality of kernels.

12. The system of claim 11, wherein the plurality of kernels in the first layer converts a plurality of data inputs into a plurality of latent variants.

13. The system of claim 12, wherein the plurality of data inputs comprise single-nucleotide polymorphisms (SNPs) or biomarkers.

14. The system of claim 11, wherein individual latent variables of the plurality of kernels are generated by random sampling of outputs of the plurality of kernels.

15. The system of claim 11, wherein the single kernel or plurality of kernels of the last layer determines the output indication based upon a plurality of latent variable produced by a preceding layer of the plurality of layers.

16. The system of claim 15, wherein the preceding layer is the first layer.

17. The system of claim 10, wherein the KNN is trained using minimum norm quadratic estimation.

18. The system of claim 10, wherein training of the KNN is accelerated using batch training.

19. The system of claim 10, wherein the training set of data and the trained KNN model are stored in a data store.

20. A non-transitory computer-readable medium embodying a program executable in at least one computing device, where when executed the program causes the at least computing device to at least:
train a kernel-based neural network (KNN) with a training set of data to produce a trained KNN model, the KNN model comprising a plurality of kernels as a plurality of layers to capture complexity between the data with disease phenotypes, the training set of data comprising genetic information applied as inputs to the KNN and one or more phenotypes;
determine a likelihood of a condition based at least in part upon an output indication of the trained KNN corresponding to the one or more phenotypes, the output indication based upon analysis of data comprising genetic information from an individual by the trained KNN; and
identify a treatment or prevention strategy for the individual based at least in part upon the likelihood of the condition.
